(19) United States
(12) Patent Application Publication
(10) Pub. No.: US 2025/0312406 A1
(43) Pub. Date: Oct. 9, 2025

(54) TRADITIONAL CHINESE MEDICINE COMPOUND EXTRACT FOR TREATING ALZHEIMER'S DISEASE AND PREPARATION METHOD THEREOF

(71) Applicant: QINGDAO CHENLAND PHARMACEUTICAL CO., LTD, QINGDAO (CN)

(72) Inventors: SHENGCAN ZOU, QINGDAO (CN); YILEI WANG, QINGDAO (CN); JIANCHENG ZONG, QINGDAO (CN); XUE WANG, QINGDAO (CN); DONGLI YIN, QINGDAO (CN); TINGTING LIU, QINGDAO (CN); NAN WANG, QINGDAO (CN)

(21) Appl. No.: 18/625,668

(22) Filed: Apr. 3, 2024

(51) Int. Cl. A61K 36/64; A61K 36/076; A61K 36/258; A61K 36/537; A61K 36/69; A61K 36/882

(52) U.S. Cl. CPC A61K 36/64; A61K 36/076; A61K 36/258; A61K 36/537; A61K 36/69; A61K 36/882; A61K 2236/15; A61K 2236/331; A61K 2236/333; A61K 2236/39; A61K 2236/51

(57) ABSTRACT

The present invention discloses a traditional Chinese medicine compound extract for treating Alzheimer's disease, including 4-10 parts of ginseng, 6-12 parts of Cistanche deserticola, 4-10 parts of Salvia miltiorrhiza, 6-12 parts of Radix polygalae, 4-10 parts of Rhizoma acori tatarinowii, 6-12 parts of dried rehmannia root and 8-16 parts of Poria cocos. A preparation method of the compound extract includes: (1) weighing; (2) alcohol extraction; (3) extraction and inclusion of volatile oil; (4) water extraction; (5) alcohol precipitation; (6) concentrating and drying; and (7) finished product preparation. In the present invention, in combination with own characteristics of the medicinal materials, various active ingredients are fully extracted. According to the preferred prescription and process through an in-vitro biological effect, a medicine for effectively treating the Alzheimer's disease is invented.

### TECHNICAL FIELD

The present invention relates to the technical field of preparation of natural medicines, and more particularly to a traditional Chinese medicine compound extract for treating Alzheimer's disease and a preparation method thereof.

### BACKGROUND

Alzheimer's disease (AD) is a degenerative disease of the central nervous system with major characteristics of cognitive function degeneration and progressive memory impairment accompanied by severe decrease of social behavior capacity and activity of daily living. Clinical manifestations of the AD include memory decline, speech and mood disorder, thought retardation and personality changes. The AD has insidious onset and slow course and has become a major disease that severely threatens human health in contemporary society, particularly quality of life of the elderly; and an incidence rate of the AD is related to increase of age.

There is no disease name of the AD in traditional Chinese medicine. The AD often belongs to the scope of “dementia”, “amnesia” and “dullness”. From the view of traditional Chinese medicine, etiology and pathogenesis of the AD are deficient root and excessive superficial symptom; the root is organ and essence deficiency; and the superficial symptom is phlegm obstructing. Therefore, the basis of treating the AD is tonifying deficiency and curing the symptoms. Through syndrome differentiation and treatment in Chinese medicine, the heart, liver, spleen and kidney are often associated. It is considered that, spirit dystrophy is caused due to deficiency of qi and blood; cerebral pulse obstruction is caused due to extravasated blood obstructing; orifice obstruction is caused due to the syndrome of phlegm turbidity; brain dystrophy is caused due to kidney essence deficiency. However, pathogenesis of the AD is not clear yet; there are few medicines in the market; and most of the medicines can mainly improve clinical symptoms of the AD.

At present, clinical biological medicines for treating the AD are divided into five classes: the first class of anticholinesterase medicines, such as galanthamine, donepezil (Aricept), huperzine A and Rivastigmine tartrate; the second class of excitatory amino acid antagonists such as memantine; the third class of amyloid β-protein antibodies such as Aducanumab; the fourth class of M1 receptor antagonists and antioxidants; and the fifth class of intestinal flora and neuroinflammation, such as GV971. However, these medicines cannot fundamentally treat the AD, but just slow the progression of the disease, while a Chinese patent medicine for treating the AD is not available in the market yet.

Therefore, how to provide a traditional Chinese medicine compound extract for treating Alzheimer's disease and a preparation method thereof is a problem that urgently needs to be solved by those skilled in the art.

### SUMMARY

In view of this, a purpose of the present invention is to provide a traditional Chinese medicine compound extract for treating Alzheimer's disease and a preparation method thereof, for solving defects in the prior art.

To achieve the above purpose, technical solutions of the present invention are as follows:

The traditional Chinese medicine compound extract for treating Alzheimer's disease includes the following raw materials in parts by weight: 4-10 parts of ginseng, 6-12 parts of Cistanche deserticola, 4-10 parts of Salvia miltiorrhiza, 6-12 parts of Radix polygalae, 4-10 parts of Rhizoma acori tatarinowii, 6-12 parts of dried rehmannia root and 8-16 parts of Poria cocos.

Preferably, the compound extract includes 6 parts of ginseng, 10 parts of Cistanche deserticola, 6 parts of Salvia miltiorrhiza, 10 parts of Radix polygalae, 6 parts of Rhizoma acori tatarinowii, 10 parts of dried rehmannia root and 12 parts of Poria cocos.

Compatibility principles of the traditional Chinese medicine compound extract in the present invention are as follows: the dried rehmannia root, the Cistanche deserticola and the Radix polygalae have effects of nourishing kidney essence; the ginseng and the Poria cocos have effects of tonifying spleen and qi; the Rhizoma acori tatarinowii has effects of expelling phlegm and clearing orifices; the Salvia miltiorrhiza has effects of promoting blood circulation to remove meridian obstruction and cooling blood; and the whole formula has precise and appropriate compatibility and achieves effects of replenishing qi to invigorate the spleen, tonifying kidney and supplementing marrow and promoting blood circulation to remove blood stasis.

Sheng Nong's Herbal Classic is the earliest existing monograph of traditional Chinese medicine, and records medicinal essence of the ginseng formed 4000 years ago as follows: “the ginseng is sweet and slightly cold in taste, has major effects of tonifying the five organs, soothing the nerves, calming the spirit, checking palpitation, dispelling pathogenic qi, improving eyesight and energizing and improving intelligence, and further achieves effects of reducing body weight and prolonging life when taken for a long time”.

The Cistanche deserticola has a use history of more than 2000 years in China. Modern pharmacological study shows that, in addition to effects of invigorating the kidney and strengthening yang, resisting aging, enhancing memory and enhancing immunity, the Cistanche deserticola ranks only second to the ginseng.

The Salvia miltiorrhiza is first recorded in Sheng Nong's Herbal Classic, is listed as a top grade medicine, and is recorded in herbal classics of successive dynasties. The modern pharmacological study shows that, the Salvia miltiorrhiza may be used for treating multiple diseases, including cerebrovascular diseases, coronary heart disease, Parkinson's disease, AD, kidney deficiency, liver cirrhosis, osteoporosis and cancers.

Compatible with the Rhizoma acori tatarinowii, the Radix polygalae is often used in a basic structure of a traditional Chinese medicine prescription for treating senile dementia in traditional Chinese medicine. Major medicinal composition in the compatible medicine refers to tenuigenin contained in the Radix polygalae and volatile oil and other components in the Rhizoma acori tatarinowii. The Radix polygalae and the Rhizoma acori tatarinowii achieve excellent effects when compatible with each other; the Rhizoma acori tatarinowii has effects of eliminating phlegm and inducing resuscitation; and the Radix polygalae has effects of eliminating phlegm and inducing resuscitation and soothing the nerves and improving intelligence. The Rhizoma acori tatarinowii is acrid in taste and achieves effects of removing phlegm-dampness; and the Radix polygalae is bitter in taste and achieves effects of reliving obstructive phlegm. Through the compatibility, the Radix polygalae and the Rhizoma acori tatarinowii enhance effects of freeing the orifice of the heart, promoting coordination between heart and kidney, tonifying the kidney, invigorating the brain, improving intelligence, inducing resuscitation and calming the mind.

It is recorded in the Sheng Nong's Herbal Classic that “the rehmannia root is sweet in taste and cold in nature, has major effects of treating external injury and internal organ injury, relieving blood-arthralgia, supplementing bone marrow and building muscles, and achieves effects of relieving accumulated chill and fever and eliminating arthralgia when prepared into decoction; fresh rehmannia root has particular effects; and the rehmannia root has effects of reducing body weight and resisting aging when taken for a long time”.

The Poria cocos has an effect of improving intelligence. It is recorded in Compendium of Materia Medica that the Poria cocos has effects of “improving mood and intelligence and preventing amnesia”. It is recorded in Classified Materia Medica that the Poria cocos can achieve effects of “nourishing yin and tonifying qi, inducing resuscitation and calming the mind, and can achieve effects of soothing the nerves, inducing resuscitation, preventing hunger and prolonging life when taken for a long time”.

The above traditional Chinese medicines have multipath, multi-target and multilevel characteristics in treatment.

The preparation method of the traditional Chinese medicine compound extract for treating Alzheimer's disease specifically includes the following steps:
(1) weighing
weighing various raw materials according to weight parts of the traditional Chinese medicine compound extract for treating Alzheimer's disease;
(2) alcohol extraction
mixing ginseng, Cistanche deserticola, Salvia miltiorrhiza and Radix polygalae; adding ethanol having a mass concentration of 50-80%; performing heating reflux extraction for 1-3 times for 1-3 h each time; collecting alcohol extraction medicine liquid and alcohol extraction medicine residue respectively; and concentrating the alcohol extraction medicine liquid to obtain an alcohol extraction concentrated solution for later use;
(3) extraction and inclusion of volatile oil
soaking Rhizoma acori tatarinowii in water for 1-4 h; then extracting volatile oil for 1-10 h; collecting Rhizoma acori tatarinowii volatile oil, a Rhizoma acori tatarinowii extracting solution and Rhizoma acori tatarinowii medicine residue respectively for later use; adding the Rhizoma acori tatarinowii volatile oil into ethanol and dissolving to obtain an oil-ethanol mixture for later use; adding hydroxypropyl-beta-cyclodextrin into water and uniformly mixing; then slowly dropwise adding the oil-ethanol mixture into the solution; performing ball milling or colloid milling; and performing vacuum drying to obtain a Rhizoma acori tatarinowii inclusion compound for later use;
(4) water extraction
mixing the alcohol extraction medicine residue, the Rhizoma acori tatarinowii medicine residue, dried rehmannia root and Poria cocos; adding water into the mixture; performing heating reflux extraction for 1-3 times for 1-3 h each time; collecting water extraction medicine liquid; concentrating the water extraction medicine liquid and the Rhizoma acori tatarinowii extracting solution; and placing the concentrated solution at room temperature to obtain water extraction concentrated solution;
(5) alcohol precipitation
adding ethanol into the water extraction concentrated solution for alcohol precipitation; refrigerating and centrifuging the solution; taking the supernatant; and concentrating the solution again to obtain a water extraction and alcohol precipitation concentrated solution for later use;
(6) concentrating and drying
mixing the alcohol extraction concentrated solution and the water extraction and alcohol precipitation concentrated solution; continuously concentrating the mixed solution; and performing vacuum drying or spray drying to obtain a mixed extract for later use;
(7) finished product preparation
uniformly mixing the Rhizoma acori tatarinowii inclusion compound and the mixed extract; grinding and sieving the mixture; adding an excipient into the mixture to make a preparation, thereby obtaining the medicine for treating the Alzheimer's disease.

Types of the preparation include pills, tablets, granules, decoction and other related traditional Chinese medicine preparations.

Further, in the step (2), the mass concentration of the ethanol is 60%; the frequency of heating reflux extraction is twice; an addition amount of the ethanol each time is 8 times that of the weight; heating reflux extraction time for each time is 2 h; a concentration temperature is 55-75° C.; and specific gravity of the concentrated solution is 1.05-1.10/50° C.

Further, in the step (3), an addition amount of the water is 8 times that of the weight; soaking time is 2 h; volatile oil extraction time is 6 h; a volume ratio of the Rhizoma acori tatarinowii extracting solution to the ethanol is 1:1; a mass ratio of the hydroxypropyl-beta-cyclodextrin to the water to the oil-ethanol mixture is 15:7.5:1; ball milling or colloid milling time is 1 h; and a vacuum drying temperature is 40° C.

Further, in the step (4), the frequency of heating reflux extraction is twice; an addition amount of the water each time is 6 times that of the weight; heating reflux extraction time for each time is 1 h; a concentration temperature is 60-90° C.; and specific gravity of the concentrated solution is 1.05-1.10/60° C.

Further, in the step (5), the ethanol is added until alcohol content is 60%; the solution is continuously concentrated until specific gravity of the concentrated solution is 1.05-1.10/60° C.; and refrigeration time is 24 h.

Further, in the step (6), the solution is continuously concentrated until specific gravity of the concentrated solution is 1.10-1.15/60° C.; a vacuum drying temperature is 40-50° C.; and the temperature of a heating tube for spray drying is 120-130° C.

Further, in the step (7), dosage forms of the traditional Chinese medicine compound extract for treating Alzheimer's disease in the present invention include pills, tablets, granules, decoction and other related traditional Chinese medicine preparations, preferably the granules. Specifically, an appropriate amount of the traditional Chinese medicine compound extract is taken; a certain amount of dextrin is added into the extract; the mixture is sprayed with ethanol to be prepared into a soft material, wherein the soft material is appropriate “when kneaded into dough and loosened into powder”; and the soft material is granulated, dried and sieved, thereby obtaining the finished product.

Through the above technical solutions, compared with the prior art, the present invention has beneficial effects as follows:
1. In the present invention, in combination with own characteristics of the medicinal materials, various active ingredients are fully extracted. According to the preferred prescription and process through an in-vitro biological effect, a medicine for effectively treating the Alzheimer's disease is invented. Through experiments, it is discovered that the traditional Chinese medicine compound extract in the present invention has a significant inhibiting effect on acetylcholin esterase (AchE).
2. In the present invention, various critical process parameters in the technical process are screened by utilizing an AchE inhibitor in-vitro screening model, for example, a four-factor three-level orthogonal test is designed, and effects of solvent concentrations, solvent doubling, extraction times and extraction time on the process in the alcohol extraction part are investigated. An absorbance value is detected by a microplate reader; an inhibition ratio is calculated; and the optimal process parameter level is obtained through orthogonal test calculation.
3. In the present invention, an effect of the traditional Chinese medicine compound extract for improving the cognitive function is observed by utilizing a learning and memorizing triplet (Morris water maze+object recognition+step-down test) mouse behavior model.

### DESCRIPTION OF DRAWINGS

FIG. 1 is a chromatogram map of ginsenoside Rg1, Re and Rb1;

FIG. 2 is a chromatogram map of tanshinone IIA and danshinolic acid B;

FIG. 3 is a chromatogram map of echinacoside and 3,6′-disinapoyl sucrose;

FIG. 4 is a chromatogram map of catalpol;

FIG. 5 is a fingerprint chromatogram;

FIG. 6 is an AchE inhibition ratio of each group in different extraction processes (notes: compared with a medicine group in the present invention, #p<0.05);

FIG. 7 is an AchE inhibition ratio of each group at different medicinal material amounts (notes: compared with a medicine group in the present invention, #p<0.05);

FIG. 8 is an AchE inhibition ratio of each group in different components (notes: compared with a medicine group in the present invention, #p21 0.05);

FIG. 9 is an AchE inhibition ratio of each group in different medicines (notes: compared with a medicine group in the present invention, #p<0.05);

FIG. 10 is an effect of a medicine in the present invention on an object recognition behavior of mice (notes: compared with a control group, *p<0.05; and compared with a model group, #p<0.05);

FIG. 11 is an effect of a medicine in the present invention on latency in a mouse step-down test (notes: compared with a control group, *p<0.05; and compared with a model group, #p<0.05);

FIG. 12 is an effect of a medicine in the present invention on error times in a mouse step-down test (notes: compared with a control group, *p<0.05; and compared with a model group, #p<0.05);

FIG. 13 is an effect of a medicine in the present invention on escape latency in a training period of a mouse water maze test;

FIG. 14 is an effect of a medicine in the present invention on escape latency in an exploration period of a mouse water maze test (compared with a control group, *p<0.05; and compared with a model group, #p<0.05);

FIG. 15 is an effect of a medicine in the present invention on target quadrant retention time in an exploration period of a mouse water maze test (compared with a control group, *p<0.05; and compared with a model group, #p<0.05);

FIG. 16 is an effect of a medicine in the present invention on a target quadrant path in an exploration period of a mouse water maze test;

FIG. 17 is an effect of a medicine in the present invention on the number of platform crossing times in an exploration period of a mouse water maze test (notes: compared with a control group, *p<0.05; and compared with a model group, #p<0.05).

### DETAILED DESCRIPTION

Technical solutions in the embodiments of the present invention are described clearly and fully below. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

In embodiments below, ginseng refers to ginseng slices; and Cistanche deserticola refers to Cistanche tubulosa slices.

### Embodiment 1

A traditional Chinese medicine compound extract for treating Alzheimer's disease includes the following raw materials by weight: 4 g of ginseng, 12 g of Cistanche deserticola, 4 g of Salvia miltiorrhiza, 12 g of Radix polygalae, 4 g of Rhizoma acori tatarinowii, 12 g of dried rehmannia root and 8 g of Poria cocos.

A preparation method of the traditional Chinese medicine compound extract for treating Alzheimer's disease specifically includes the following steps:
(1) weighing
weighing various raw materials according to the weight of the traditional Chinese medicine compound extract for treating Alzheimer's disease;
(2) alcohol extraction
mixing ginseng, Cistanche deserticola, Salvia miltiorrhiza and Radix polygalae; adding ethanol in an amount of 8 times that of the weight having a mass concentration of 60%; performing heating reflux extraction twice; performing extraction for 2 h each time; collecting alcohol extraction medicine liquid and alcohol extraction medicine residue respectively; and concentrating the alcohol extraction medicine liquid at 55-75° C. to obtain an alcohol extraction concentrated solution having specific gravity of 1.05/50° C. for later use;
(3) extraction and inclusion of volatile oil
soaking Rhizoma acori tatarinowii in water in an amount of 8 times that of the weight for 2 h; then extracting volatile oil for 6 h; collecting Rhizoma acori tatarinowii volatile oil, a Rhizoma acori tatarinowii extracting solution and Rhizoma acori tatarinowii medicine residue respectively for later use; adding the Rhizoma acori tatarinowii volatile oil into ethanol of the same volume and dissolving to obtain an oil-ethanol mixture for later use; adding hydroxypropyl-beta-cyclodextrin into water and uniformly mixing; then slowly dropwise adding the oil-ethanol mixture into the solution, wherein a mass ratio of the hydroxypropyl-beta-cyclodextrin to the water to the oil-ethanol mixture is 15:7.5:1; performing ball milling for 1 h; and then performing vacuum drying at a temperature of 40° C. to obtain a Rhizoma acori tatarinowii inclusion compound for later use;
(4) water extraction
mixing the alcohol extraction medicine residue, the Rhizoma acori tatarinowii medicine residue, dried rehmannia root and Poria cocos; adding water in an amount of 6 times that of the weight into the mixture; performing heating reflux extraction twice; performing extraction for 1 h each time; collecting water extraction medicine liquid; concentrating the water extraction medicine liquid and the Rhizoma acori tatarinowii extracting solution at 60-90° C. until specific gravity of the concentrated solution is 1.05/60° C.; and placing the concentrated solution at room temperature to obtain water extraction concentrated solution;
(5) alcohol precipitation
adding ethanol into the water extraction concentrated solution until alcohol content is 60% for alcohol precipitation; performing closed refrigeration for 24 h; centrifuging the solution; taking the supernatant; and concentrating the solution again until specific gravity of the concentrated solution is 1.05/60° C. to obtain a water extraction and alcohol precipitation concentrated solution for later use;
(6) mixing
mixing the alcohol extraction concentrated solution and the water extraction and alcohol precipitation concentrated solution; continuously concentrating the mixed solution until specific gravity of the concentrated solution is 1.10/60° C.; and performing vacuum drying at 50° C. obtain a mixed extract for later use;
(7) finished product preparation
uniformly mixing the Rhizoma acori tatarinowii inclusion compound and the mixed extract; grinding and sieving the mixture to obtain a traditional Chinese medicine compound extract; then adding dextrin; spraying the mixture with ethanol to be prepared into a soft material, wherein the soft material is appropriate “when kneaded into dough and loosened into powder”; and granulating, drying and sieving the soft material, thereby obtaining the traditional Chinese medicine compound granules for treating the Alzheimer's disease.

### Embodiment 2

A traditional Chinese medicine compound extract for treating Alzheimer's disease includes the following raw materials by weight: 6 g of ginseng, 10 g of Cistanche deserticola, 6 g of Salvia miltiorrhiza, 10 g of Radix polygalae, 6 g of Rhizoma acori tatarinowii, 10 g of dried rehmannia root and 12 g of Poria cocos.

A preparation method of the traditional Chinese medicine compound extract for treating Alzheimer's disease specifically includes the following steps:
(1) weighing
weighing various raw materials according to the weight of the traditional Chinese medicine compound extract for treating Alzheimer's disease;
(2) alcohol extraction
mixing ginseng, Cistanche deserticola, Salvia miltiorrhiza and Radix polygalae; adding ethanol in an amount of 8 times that of the weight having a mass concentration of 60%; performing heating reflux extraction twice; performing extraction for 2 h each time; collecting alcohol extraction medicine liquid and alcohol extraction medicine residue respectively; and concentrating the alcohol extraction medicine liquid at 55-75° C. to obtain an alcohol extraction concentrated solution having specific gravity of 1.08/50° C. for later use;
(3) extraction and inclusion of volatile oil
soaking Rhizoma acori tatarinowii in water in an amount of 8 times that of the weight for 2 h; then extracting volatile oil for 6 h; collecting Rhizoma acori tatarinowii volatile oil, a Rhizoma acori tatarinowii extracting solution and Rhizoma acori tatarinowii medicine residue respectively for later use; adding the Rhizoma acori tatarinowii volatile oil into ethanol of the same volume and dissolving to obtain an oil-ethanol mixture for later use; adding hydroxypropyl-beta-cyclodextrin into water and uniformly mixing; then slowly dropwise adding the oil-ethanol mixture into the solution, wherein a mass ratio of the hydroxypropyl-beta-cyclodextrin to the water to the oil-ethanol mixture is 15:7.5:1; performing ball milling for 1 h; and then performing vacuum drying at a temperature of 40° C. to obtain a Rhizoma acori tatarinowii inclusion compound for later use;
(4) water extraction
mixing the alcohol extraction medicine residue, the Rhizoma acori tatarinowii medicine residue, dried rehmannia root and Poria cocos; adding water in an amount of 6 times that of the weight into the mixture; performing heating reflux extraction twice; performing extraction for 1 h each time; collecting water extraction medicine liquid; concentrating the water extraction medicine liquid and the Rhizoma acori tatarinowii extracting solution at 60-90° C. until specific gravity of the concentrated solution is 1.08/60° C.; and placing the concentrated solution at room temperature to obtain water extraction concentrated solution;
(5) alcohol precipitation
adding ethanol into the water extraction concentrated solution until alcohol content is 60% for alcohol precipitation; performing closed refrigeration for 24 h; centrifuging the solution; taking the supernatant; and concentrating the solution again until specific gravity of the concentrated solution is 1.08/60° C. to obtain a water extraction and alcohol precipitation concentrated solution for later use;
(6) mixing
mixing the alcohol extraction concentrated solution and the water extraction and alcohol precipitation concentrated solution; continuously concentrating the mixed solution until specific gravity of the concentrated solution is 1.12/60° C.; and performing vacuum drying at 50° C. obtain a mixed extract for later use;
(7) finished product preparation
uniformly mixing the Rhizoma acori tatarinowii inclusion compound and the mixed extract; grinding and sieving the mixture to obtain a traditional Chinese medicine compound extract; then adding dextrin; spraying the mixture with ethanol to be prepared into a soft material, wherein the soft material is appropriate “when kneaded into dough and loosened into powder”; and granulating, drying and sieving the soft material, thereby obtaining the traditional Chinese medicine compound granules for treating the Alzheimer's disease.

### Embodiment 3

A traditional Chinese medicine compound extract for treating Alzheimer's disease includes the following raw materials by weight: 10 g of ginseng, 6 g of Cistanche deserticola, 10 g of Salvia miltiorrhiza, 6 g of Radix polygalae, 10 g of Rhizoma acori tatarinowii, 6 g of dried rehmannia root and 16 g of Poria cocos.

A preparation method of the traditional Chinese medicine compound extract for treating Alzheimer's disease specifically includes the following steps:
(1) weighing
weighing various raw materials according to the weight of the traditional Chinese medicine compound extract for treating Alzheimer's disease;
(2) alcohol extraction
mixing ginseng, Cistanche deserticola, Salvia miltiorrhiza and Radix polygalae; adding ethanol in an amount of 8 times that of the weight having a mass concentration of 60%; performing heating reflux extraction twice; performing extraction for 2 h each time; collecting alcohol extraction medicine liquid and alcohol extraction medicine residue respectively; and concentrating the alcohol extraction medicine liquid at 55-75° C. to obtain an alcohol extraction concentrated solution having specific gravity of 1.10/50° C. for later use;
(3) extraction and inclusion of volatile oil
soaking Rhizoma acori tatarinowii in water in an amount of 8 times that of the weight for 2 h; then extracting volatile oil for 6 h; collecting Rhizoma acori tatarinowii volatile oil, a Rhizoma acori tatarinowii extracting solution and Rhizoma acori tatarinowii medicine residue respectively for later use; adding the Rhizoma acori tatarinowii volatile oil into ethanol of the same volume and dissolving to obtain an oil-ethanol mixture for later use; adding hydroxypropyl-beta-cyclodextrin into water and uniformly mixing; then slowly dropwise adding the oil-ethanol mixture into the solution, wherein a mass ratio of the hydroxypropyl-beta-cyclodextrin to the water to the oil-ethanol mixture is 15:7.5:1; performing ball milling for 1 h; and then performing vacuum drying at a temperature of 40° C. to obtain a Rhizoma acori tatarinowii inclusion compound for later use;
(4) water extraction
mixing the alcohol extraction medicine residue, the Rhizoma acori tatarinowii medicine residue, dried rehmannia root and Poria cocos; adding water in an amount of 6 times that of the weight into the mixture; performing heating reflux extraction twice; performing extraction for 1 h each time; collecting water extraction medicine liquid; concentrating the water extraction medicine liquid and the Rhizoma acori tatarinowii extracting solution at 60-90° C. until specific gravity of the concentrated solution is 1.10/60° C.; and placing the concentrated solution at room temperature to obtain water extraction concentrated solution;
(5) alcohol precipitation
adding ethanol into the water extraction concentrated solution until alcohol content is 60% for alcohol precipitation; performing closed refrigeration for 24 h; centrifuging the solution; taking the supernatant; and concentrating the solution again until specific gravity of the concentrated solution is 1.10/60° C. to obtain a water extraction and alcohol precipitation concentrated solution for later use;
(6) mixing
mixing the alcohol extraction concentrated solution and the water extraction and alcohol precipitation concentrated solution; continuously concentrating the mixed solution until specific gravity of the concentrated solution is 1.15/60° C.; and performing vacuum drying at 50° C. obtain a mixed extract for later use;
(7) finished product preparation
uniformly mixing the Rhizoma acori tatarinowii inclusion compound and the mixed extract; grinding and sieving the mixture to obtain a traditional Chinese medicine compound extract; then adding dextrin; spraying the mixture with ethanol to be prepared into a soft material, wherein the soft material is appropriate “when kneaded into dough and loosened into powder”; and granulating, drying and sieving the soft material, thereby obtaining the traditional Chinese medicine compound granules for treating the Alzheimer's disease.

### Embodiment 4

Differences from embodiment 2 are only as follows: “ball milling for 1 h” in the step (3) is replaced with “colloid milling for 1 h”; and “performing vacuum drying at 50° C.” in the step (6) is replaced with “performing spray drying at 125° C.”.

### Embodiment 5

The difference from embodiment 2 is only as follows: the step (7) is replaced with a step of “uniformly mixing the Rhizoma acori tatarinowii inclusion compound and the mixed extract; grinding and sieving the mixture to obtain a traditional Chinese medicine compound extract; then adding dextrin; spraying the mixture with ethanol to be prepared into a soft material, wherein the soft material is appropriate “when kneaded into dough and loosened into powder”; granulating, drying and sieving the soft material; adding 0.5% of magnesium stearate; uniformly mixing the soft material and magnesium stearate; and pressing the mixture into tablets, thereby obtaining the traditional Chinese medicine compound tablets for treating the Alzheimer's disease”.

### PERFORMANCE TEST

### I. Quality Standards

### 1. Establishment of Quality Standards of the Extract

### 1.1 Content

The ginseng refers to ginsenoside Rg1, Re and Rb1; the Cistanche deserticola refers to echinacoside; the Salvia miltiorrhiza refers to tanshinone IIA and danshinolic acid B; the Radix polygalae refers to 3,6′-disinapoyl sucrose; and the dried rehmannia root refers to catalpol.

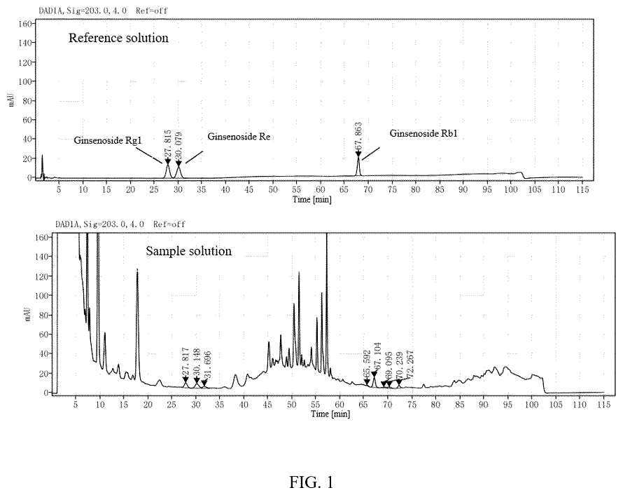

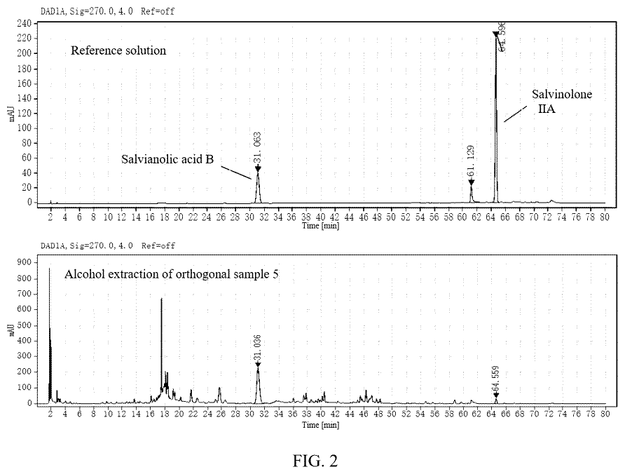

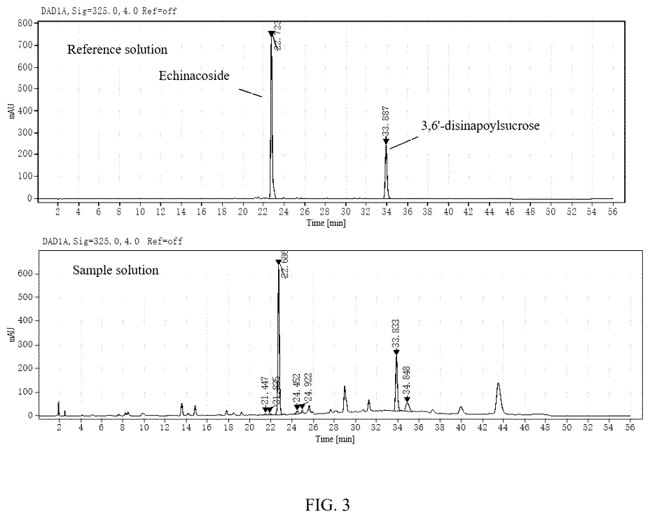

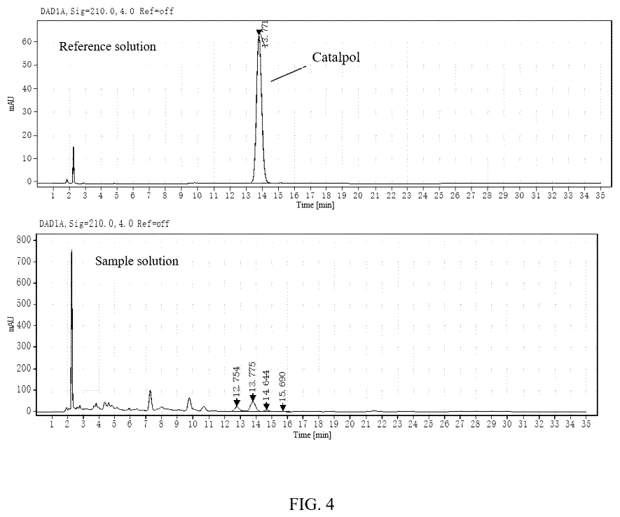

The above content is determined by high performance liquid chromatography; and through method development and methodology validation, a detection method is determined. Detection chromatograms are shown as FIGS. 1-4.

### 1.2 Fingerprint Chromatogram

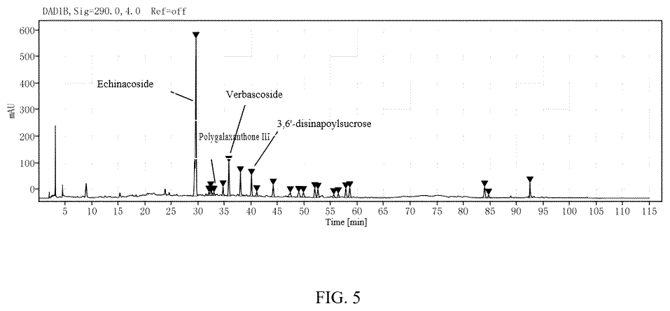

Fingerprint chromatogram detection conditions are established by high performance liquid chromatography. Preferably, echinacoside, 3,6′-disinapoyl sucrose, verbascoside and polygalaxanthone III mainly serve as qualitative components; and the established fingerprint chromatogram is mainly used for serving as one of the quality control methods for rapidly detecting the extract during future commercial production. The fingerprint chromatogram is shown as FIG. 5.

### II. Pharmaceutical Effect

### 1. In-Vitro Inhibiting Effects of Samples in Different Extraction Processes on Acetylcholin Esterase

### 1.1 Materials

The medicine in the present invention (mixed extract prepared in the step (6) of embodiment 2), a water extraction sample, an alcohol extraction sample, acetylcholin esterase (AchE, Shanghai Yuanye Bio-Technology Co., Ltd.), huperzine A (HPLC≥98%, Shanghai Yuanye Bio-Technology Co., Ltd.), and the like.

### 1.2 Preparation of Samples

### 1.2.1 Water Extract

6 g of ginseng, 10 g of Cistanche deserticola, 6 g of Salvia miltiorrhiza, 10 g of Radix polygalae, 6 g of Rhizoma acori tatarinowii, 10 g of dried rehmannia root and 12 g of Poria cocos were added into a round-bottom flask; water in an amount of 8 times that of the weight was added; extraction was performed twice for 2 h each time; an extracting solution was collected; the extracting solution was concentrated to 1.10-1.15/60° C.; and vacuum drying was conducted at 50° C. to obtain an extract, thereby obtaining the water extract sample.

### 1.2.2 Alcohol Extract

6 g of ginseng, 10 g of Cistanche deserticola, 6 g of Salvia miltiorrhiza, 10 g of Radix polygalae, 6 g of Rhizoma acori tatarinowii, 10 g of dried rehmannia root and 12 g of Poria cocos were added into a round-bottom flask; ethanol in an amount of 8 times that of the weight was added; extraction was performed twice for 2 h each time; an extracting solution was collected; the extracting solution was concentrated to 1.10-1.15/50° C.; and vacuum drying was conducted at 50° C. to obtain an extract, thereby obtaining the alcohol extract sample.

### 1.2.3 Medicine in the Present Invention

The mixed extract prepared in the step (6) of embodiment 2.

### 1.2.4 Sample Concentration

A final concentration of the system is 1 mg/mL.

### 1.3 Test Method

The test method is an improved Ellman method. A basic principle is as follows: acetylthiocholine iodide (ATCI) serves as a substrate; after AChE is added, the ATCI will be hydrolyzed in presence of the AChE to form thiocholine; the thiocholine reacts with DTNB to produce 2-nitrobenzoic acid-5-thiocholine and 2-nitro-5-thiosalicylic acid (TNB); and an absorbance value of yellow ions (TNB) may be detected at 412 nm. During determination of inhibitory activity of a plant sample on AChE, if the plant sample has an inhibiting effect on the AChE, an amount of the ATCI in the reaction is decreased, and thus the produced produce (thiocholine) is decreased. Moreover, the amount of the product reacting with DTNB is also correspondingly decreased, finally production of the TNB is decreased, and then the absorbance value is decreased at 412 nm. On the contrary, the absorbance value is increased at 412 nm.

### 1.4 Measurement Index

Absorbance value of each compound pore

### 1.5 Test Result

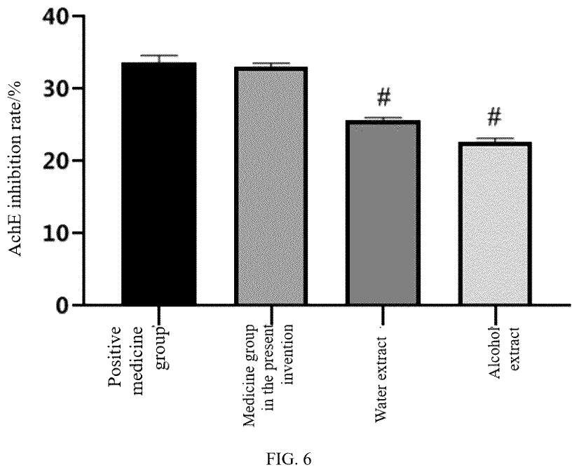

The obtained result is represented by mean±standard deviation; statistical analysis is conducted by SPSS26.0; analysis of variance (ANOVA) is adopted among groups; p<0.05 shows significance of difference. The result is shown as Table 1 and FIG. 6.

TABLE 1
AchE inhibition ratio of each group
in different extraction processes

| | Parallel | Parallel | Parallel | |
|---|---|---|---|---|
| Group | sample 1 | sample 2 | sample 3 | Mean |
| Huperzine A group | 34.70% | 33.50% | 32.94% | 33.7% ± 0.9% |
| Medicine group in | 32.50% | 33.10% | 33.46% | 33.0% ± 0.5% |
| the present | | | | |
| invention | | | | |
| Water extract | 26.02% | 25.47% | 25.69% | 25.7% ± 0.3% |
| Alcohol extract | 22.13% | 23.10% | 22.54% | 22.6% ± 0.5% |

### 1.6 Discussion

During cholinergic synapse, the acetylcholin esterase can degrade acetyl choline, terminate an excitatory action of neurotransmitters at the postsynaptic membrane and ensure normal transmission of neural signals in vivo. Researches show that, basal forebrain of an AD patient has cholinergic nerve cell loss; activity of the AchE is increased, while content of the acetyl choline serving as a neurotransmitter is decreased. Therefore, with respect to the hypothesis, inhibiting the activity of the AchE by taking the AchE as a target is a classical strategy of treating the AD.

In the present experimental result, the inhibiting effect of the medicine in the present invention on the AchE is obviously better than that of the water extract and the alcohol extract, and has significant difference, which indicates that the process adopted by the medicine in the present invention is better than the traditional water extraction process and alcohol extraction process.

### 2. In-Vitro Inhibiting Effects of Samples of Different Medicinal Material Amounts on Acetylcholin Esterase

### 2.1 Materials

The medicine in the present invention (traditional Chinese medicine compound granules prepared in the step (7) of embodiment 2), sample 1 (traditional Chinese medicine compound granules prepared in the step (7) of embodiment 1), sample 2 (traditional Chinese medicine compound granules prepared in the step (7) of embodiment 3), acetylcholin esterase (AchE, Shanghai Yuanye Bio-Technology Co., Ltd.), huperzine A (HPLC≥98%, Shanghai Yuanye Bio-Technology Co., Ltd.), and the like.

### 2.2 Sample Concentration

A final concentration of the system is 1 mg/mL.

### 2.3 Test Method

The test method is an improved Ellman method. A basic principle is as follows: acetylthiocholine iodide (ATCI) serves as a substrate; after AChE is added, the ATCI will be hydrolyzed in presence of the AChE to form thiocholine; the thiocholine reacts with DTNB to produce 2-nitrobenzoic acid-5-thiocholine and 2-nitro-5-thiosalicylic acid (TNB); and an absorbance value of yellow ions (TNB) may be detected at 412 nm. During determination of inhibitory activity of a plant sample on AChE, if the plant sample has an inhibiting effect on the AChE, an amount of the ATCI in the reaction is decreased, and thus the produced produce (thiocholine) is decreased. Moreover, the amount of the product reacting with DTNB is also correspondingly decreased, finally production of the TNB is decreased, and then the absorbance value is decreased at 412 nm. On the contrary, the absorbance value is increased at 412 nm.

### 2.4 Measurement Index

Absorbance value of each compound pore

### 2.5 Test Result

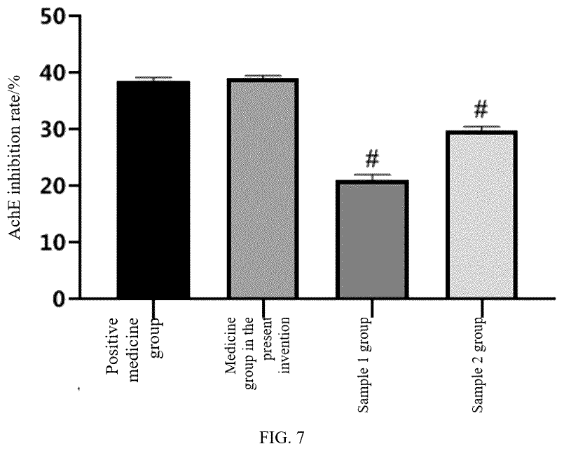

The obtained result is represented by mean±standard deviation; statistical analysis is conducted by SPSS26.0; analysis of variance (ANOVA) is adopted among groups; and p<0.05 shows significance of difference. The result is shown as Table 2 and FIG. 7.

TABLE 2
AchE inhibition ratio of each group at
different medicinal material amounts

| | Parallel | Parallel | Parallel | |
|---|---|---|---|---|
| Group | sample 1 | sample 2 | sample 3 | Mean |
| Huperzine A group | 38.15% | 39.24% | 38.53% | 38.6% ± 0.6% |
| Medicine group in | 39.11% | 39.34% | 38.47% | 39.0% ± 0.5% |
| the present | | | | |
| invention | | | | |
| Sample 1 group | 21.02% | 20.49% | 21.98% | 21.2% ± 0.8% |
| Sample 2 group | 29.15% | 29.72% | 30.60% | 29.8% ± 0.7% |

### 2.6 Discussion

Identical medicinal slices are prepared, and inhibiting effects of three samples of different amounts on the AchE are compared. For the results, the inhibiting effect of the medicine in the present invention on the AchE is obviously better than that of the sample 1 group and the sample 2 group; and the inhibiting effects of the three samples on the AchE have significant differences, which indicates that the medicine composition in the present invention is better than other compositions under different composition conditions.

### 3. In-Vitro Inhibiting Effects of Different Compositions on Acetylcholin Esterase

### 3.1 Materials

The medicine in the present invention (mixed extract prepared in the step (6) of embodiment 2), sample 1 (self-prepared), sample 2 (self-prepared), sample 3 (self-prepared), acetylcholin esterase (AchE, Shanghai Yuanye Bio-Technology Co., Ltd.), huperzine A (HPLC≥98%, Shanghai Yuanye Bio-Technology Co., Ltd.), and the like.

### 3.2 Preparation of Samples

### 3.2.1 Medicine in the Present Invention

The mixed extract prepared in the step (6) of embodiment 2.

### 3.2.2 Sample 1

60 g of ginseng, 100 g of Radix polygalae, 60 g of Rhizoma acori tatarinowii, 120 g of Poria cocos and 100 g of dried rehmannia root are respectively extracted and prepared according to embodiment 2, thereby obtaining the sample 1.

### 3.2.3 Sample 2

60 g of ginseng, 100 g of Radix polygalae, 60 g of Salvia miltiorrhiza, 60 g of Rhizoma acori tatarinowii, 120 g of Poria cocos and 100 g of dried rehmannia root are respectively extracted and prepared according to embodiment 2, thereby obtaining the sample 2 .

### 3.2.4 Sample 3

60 g of ginseng, 100 g of Radix polygalae, 100 g of Cistanche deserticola, 60 g of Rhizoma acori tatarinowii, 120 g of Poria cocos and 100 g of dried rehmannia root are respectively extracted and prepared according to embodiment 2, thereby obtaining the sample 3.

### 3.2.5 Sample Concentration

A final concentration of the system is 1 mg/mL.

### 3.3 Test Method

The test method is an improved Ellman method. A basic principle is as follows: acetylthiocholine iodide (ATCI) serves as a substrate; after AChE is added, the ATCI will be hydrolyzed in presence of the AChE to form thiocholine; the thiocholine reacts with DTNB to produce 2-nitrobenzoic acid-5-thiocholine and 2-nitro-5-thiosalicylic acid (TNB); and an absorbance value of yellow ions (TNB) may be detected at 412 nm. During determination of inhibitory activity of a plant sample on AChE, if the plant sample has an inhibiting effect on the AChE, an amount of the ATCI in the reaction is decreased, and thus the produced produce (thiocholine) is decreased. Moreover, the amount of the product reacting with DTNB is also correspondingly decreased, finally production of the TNB is decreased, and then the absorbance value is decreased at 412 nm. On the contrary, the absorbance value is increased at 412 nm.

### 3.4 Measurement Index

Absorbance value of each compound pore

### 3.5 Test Result

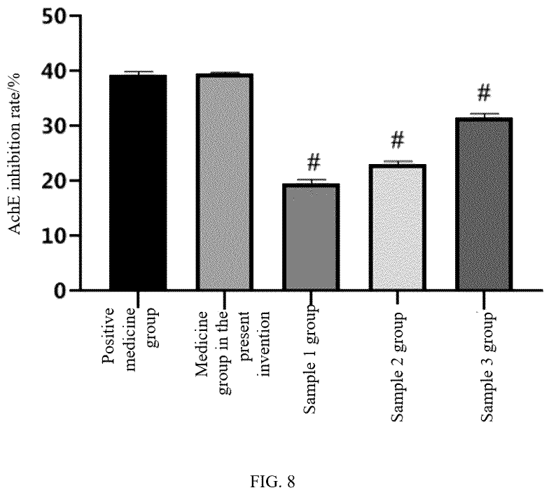

The obtained result is represented by mean±standard deviation; statistical analysis is conducted by SPSS26.0; analysis of variance (ANOVA) is adopted among groups; p<0.05 shows significance of difference. The result is shown as Table 3 and FIG. 8.

TABLE 3
AchE inhibition ratio of each group at different compositions

| | Parallel | Parallel | Parallel | |
|---|---|---|---|---|
| Group | sample 1 | sample 2 | sample 3 | Mean |
| Huperzine A group | 39.41% | 39.78% | 38.83% | 39.3% ± 0.5% |
| Medicine group in | 39.56% | 39.84% | 39.17% | 39.5% ± 0.3% |
| the present | | | | |
| invention | | | | |
| Sample 1 group | 19.02% | 19.96% | 20.11% | 19.7% ± 0.6% |
| Sample 2 group | 23.64% | 23.10% | 22.85% | 23.2% ± 0.4% |
| Sample 3 group | 32.26% | 30.89% | 31.74% | 31.6% ± 0.7% |

### 3.6 Discussion

During cholinergic synapse, the acetylcholin esterase can degrade acetyl choline, terminate an excitatory action of neurotransmitters at the postsynaptic membrane and ensure normal transmission of neural signals in vivo. Researches show that, basal forebrain of an AD patient has cholinergic nerve cell loss; activity of the AchE is increased, while content of the acetyl choline serving as a neurotransmitter is decreased. Therefore, with respect to the hypothesis, inhibiting the activity of the AchE by taking the AchE as a target is a classical strategy of treating the AD.

In the present experiment, a final concentration of the huperzine A is 70 μg/L; the final concentration of the medicine in the present invention, the sample 1, the sample 2 and the sample 3 is respectively 1 mg/mL; and the huperzine A is a cholinesterase inhibitor, has a selective inhibiting effect on the acetylcholin esterase, and easily passes through the blood-brain barrier. The huperzine A has effects of promoting memory representation, enhancing memory retention and strengthening muscle contraction. Thus, for the AchE inhibiting results, the effect of the huperzine A is significant; the inhibiting effect of the medicine in the present invention is obviously better than that of the samples 1, 2 and 3; and the difference is significant. Moreover, the inhibiting effects of the four samples on the AchE have significant differences, which indicate that the composition of the medicine in the present invention is better than the other compositions under different composition conditions.

### 4. In-Vitro Inhibiting Effects of the Medicine in the Present Invention, Kaixin Powder and Qiongyugao on Acetylcholin Esterase

### 4.1 Materials

The medicine in the present invention (traditional Chinese medicine compound granules prepared in the step (7) of embodiment 2), Kaixin powder (self-prepared), Qiongyugao (self-prepared), acetylcholin esterase (AchE, Shanghai Yuanye Bio-Technology Co., Ltd.), huperzine A (HPLC≥98%, Shanghai Yuanye Bio-Technology Co., Ltd.), and the like.

4.2 Preparation of Samples

### 4.2.1 Kaixin Powder

60 g of Radix polygalae, 60 g of ginseng, 120 g of Poria cocos and 60 g of Rhizoma acori tatarinowii were prepared into medicinal slices; water was added to immerse the medicinal slices; the medicinal slices were decocted together; decocting was performed for 45 min for the first time; the solution was filtered to obtain decoction for later use; water of the same amount was added into the medicine residue for the second time; decocting was performed for 30 min; the solution was filtered to obtain secondary decoction; the decoction and the secondary decoction were merged; vacuum concentration was performed; vacuum drying was conducted when the amount was appropriate; and the product was ground, thereby obtaining the sample.

### 4.2.2 Qiongyugao

250 g of dried rehmannia root and 100 g of Poria cocos were subjected to reflux extraction; water was respectively added in an amount of 8 times that of the weight and in an amount of 6 times that of the weight; extraction was conducted for 2 h each time; the medicine residue was filtered; the filtrate was obtained for later use; 50 g of ginseng slices were subjected to reflux extraction; water was respectively added in an amount of 8 times that of the weight and in an amount of 6 times that of the weight; extraction was conducted for 2 h each time; the filtrate was obtained; then the filtrate was merged with the filtrate of the dried rehmannia root and Poria cocos; the merged filtrate was subjected to standing for 24 h; the supernatant was concentrated into 1 L of stock solution having specific gravity of 1.23 g/mL for later use; 350 g of honey was refined into medium honey; the medium honey was added into the 1 L of stock solution; the mixed solution was continuously concentrated to 1 L until specific gravity of the concentrated solution was 1.4 g/mL.

### 4.2.3 Medicine in the Present Invention

Traditional Chinese medicine compound granules prepared in the step (7) of embodiment 2.

### 4.2.4 Sample Concentration

A final concentration of the system is 1 mg/mL.

### 4.3 Test Method

The test method is an improved Ellman method. A basic principle is as follows: acetylthiocholine iodide (ATCI) serves as a substrate; after AChE is added, the ATCI will be hydrolyzed in presence of the AChE to form thiocholine; the thiocholine reacts with DTNB to produce 2-nitrobenzoic acid-5-thiocholine and 2-nitro-5-thiosalicylic acid (TNB); and an absorbance value of yellow ions (TNB) may be detected at 412 nm. During determination of inhibitory activity of a plant sample on AChE, if the plant sample has an inhibiting effect on the AChE, an amount of the ATCI in the reaction is decreased, and thus the produced produce (thiocholine) is decreased. Moreover, the amount of the product reacting with DTNB is also correspondingly decreased, finally production of the TNB is decreased, and then the absorbance value is decreased at 412 nm. On the contrary, the absorbance value is increased at 412 nm.

### 4.4 Measurement Index

Absorbance value of each compound pore

### 4.5 Test Result

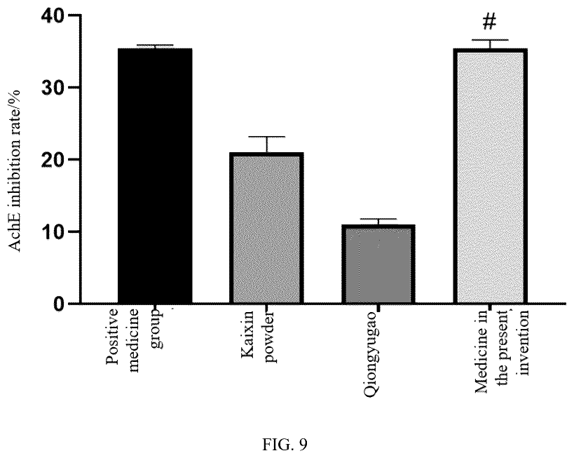

The obtained result is represented by mean±standard deviation; statistical analysis is conducted by SPSS26.0; analysis of variance (ANOVA) is adopted among groups; and p<0.05 shows significance of difference. The result is shown as Table 4 and FIG. 9.

TABLE 4
AchE inhibition ratio of each group at different medicines

| | Parallel | Parallel | Parallel | |
|---|---|---|---|---|
| Group | sample 1 | sample 2 | sample 3 | Mean |
| Huperzine A group | 35.92% | 34.94% | 35.19% | 35.4% ± 0.5% |
| Kaixin powder | 21.94% | 18.74% | 22.61% | 21.1% ± 2.1% |
| group | | | | |
| Qiongyugao group | 11.47% | 10.28% | 11.53% | 11.1% ± 0.7% |
| Medicine group in | 36.34% | 34.39% | 35.73% | 35.5% ± 1.0% |
| the present | | | | |
| invention | | | | |

### 4.6 Discussion

In the present experiment, a final concentration of the huperzine A is 70 μg/L; the concentration of the Kaixin powder, the Qiongyugao and the medicine in the present invention is respectively 1 mg/mL; and the huperzine A is a cholinesterase inhibitor, has a selective inhibiting effect on the acetylcholin esterase, and easily passes through the blood-brain barrier. The huperzine A has effects of promoting memory representation, enhancing memory retention and strengthening muscle contraction. Thus, for the AchE inhibiting results, the inhibiting effect of the huperzine A on AchE and the inhibiting effect of the medicine in the present invention on AchE are obviously better than that of the Kaixin powder and Qiongyugao groups; and the difference is significant.

### 5. Learning and Memorizing Triplet (Morris Water Maze+Object Recognition+Step-Down Test) Mouse Behavior Model

Object recognition is a behavioral method for detecting short-term memory power of mice by utilizing the nature that the mice like to approach and explore strange objects.

### 5.1 Materials

### 5.1.1 Instruments and Consumables

Object recognition experiment system: Shanghai Xinruan Information Technology Co., Ltd.;
SuperMaze animal behavior analysis system: Shanghai Xinruan Information Technology Co., Ltd.;
Water maze experiment system: Shanghai Xinruan Information Technology Co., Ltd.;
Step-down test system: Jinan Yiyan Scientific and Technological Development Co., Ltd.;
Electronic scale: Sartorius Group;
Mouse gavage apparatus: Jinan Yiyan Scientific and Technological Development Co., Ltd.

### 5.1.2 Medicine

Medicine to be tested: the medicine in the present invention (traditional Chinese medicine compound granules prepared in the step (7) of embodiment 2);
Huperzine A tablets: Henan Taloph Pharmaceutical Stock Co., Ltd.; specification: 50 μg per tablet (24 tablets per box); batch number: 210902;
Scopolamine hydrobromide tablets: J&K Scientific Co., Ltd.; specification: 1 g (bottle); batch number: LI10Q53.

### 5.1.3 Animals

Animals: 140 male SPF-grade C57 mice having body mass of 20±2g; within 7 days after adaptive feeding, there are 120 mice left, and each group includes 15 mice. The mice are provided by Jinan Pengyue Laboratory Animal Breeding Co., Ltd. with a license number of SCXK (Lu) 2018-0003.

The mice are divided into 6 groups according to a randomized block design method (according to the body mass): a normal control group; a model group; a positive medicine group; a Kaixin powder group; a Qiongyugao group; a low-dose medicine group in the present invention; a medium-dose medicine group in the present invention and a high-dose medicine group in the present invention.

### 5.2 Administration Mode and Cycle

At the fixed time every day, the mice in the normal control group and the model group are subjected to intragastric administration with distilled water of the same volume (an intragastric administration volume is 0.1 mL/10g); the Kaixin powder group (having an intragastric administration volume of 0.1 mL/10g and an administration dose of 2.5 g/kg); the Qiongyugao group (having an intragastric administration volume of 0.1 mL/10g and an administration dose of 2.5 g/kg); the medicine in the present invention at different administration concentrations is administered on the mice in the low/medium/high-dose group (a low dose of 1.25 g/kg; a medium dose of 2.5 g/kg and a high dose of 5 g/kg); and huperzine A tablets (having an intragastric administration volume of 0.1 mL/10 g and an administration dose of 26 μg/kg) are administered on the mice in the positive medicine group for 7 consecutive days. Within 60 min after the last intragastric administration, except that the mice in the normal control group are injected with 0.9% normal saline of the same volume, 2 mg/kg of scopolamine hydrobromide (a dose explored and determined in the preliminary experiment) is injected into the abdominal cavity of the mice in the rest 4 groups at an injection volume of 0.1 mL/10 g until the experiment is ended. Administration is conducted within 1 h before the object recognition, step-down and water maze tests; and the scopolamine hydrobromide and normal saline are injected into the abdominal cavity in the first 15-20 min.

### 5.3 Test Method

### 5.3.1 Novel Object Recognition Test

The novel object recognition test (NOR) is a behavioral method for detecting short-term memory power of mice by utilizing the nature that the mice like to approach and explore novel objects. Test operation procedures are as follows:

Adaptation period: on Day 1, the mice are placed in a test room to conduct environment adaptation and familiarity for 1 h; and then the mice are put in an open field test case for adaptation for 20 min (no object in the case) to eliminate an effect of the open field test case on the tested case. After each mouse is completely tested, the open field test case is cleaned with 30% ethanol.

Familiarity period: on Day 2 (within 24 h after the adaptation period), the mice are placed in the test room to conduct environment adaptation and familiarity for 1 h; then two yellow cylindrical bottles of identical size and texture are placed at the left and right ends of the same side in the open field test case to serve as familiar objects; the mice are put in the case back on the objects; object exploring conditions of the mice are recorded within 5 min (timing standard: exploration time when noses or mouths of the mice are 2 cm away from the objects; the time when the mice lean over the objects or just walk nearby the objects is not counted). After each mouse is completely tested, the open field test case is cleaned with 75% ethanol.

Test period: within 2 h after the familiarity period, one yellow cylindrical bottle in the open field test case is randomly replaced with a green conical flask of the same size to serve as a novel object; then the mice are put in the case back on the object; and the total exploration time of novel object (Tn) and the total exploration time of familiar object (Tf) of the mice are recorded within 5 min. A recognition index is calculated. After each mouse is completely tested, the open field test case is cleaned with 30% ethanol.

### 5.3.2 Step-Down Test of Mice

Within 1 d after the object recognition test is ended, step-down training is conducted for adaptation for 3 min; then electrified training is conducted for 300 s; test is conducted within 24 h; then the mice are put on a platform; and latency of the mice that jump off the platform for the first time and the number of error times (jumping off the platform) within 300 s are recorded. If the mice do not jump off the platform within 300 s, the number of error times is recorded as 0, and the latency is recorded as 300 s.

### 5.3.3 Water Maze Test of Mice

The water maze test is conducted within 1 d after the step-down test. The water maze test is divided into two parts, and conducted for 5 consecutive days. The first part is navigation test for testing the mice for 4 d; and the second part is space exploration test for testing the mice for 1 d. Edible melanin powder is mixed with warm water to hide the platform, and enabled to maintain a sharp contrast to the color of the mice during camera shooting for tracking. The platform is located at 0.8 cm below the water surface; a water depth is 21 cm; the platform is located at the NW quadrant; and the temperature of water in the water maze is maintained at a constant temperature of 22-24° C. by a heating rod.

Navigation test: each mouse is trained by 4 times per day; and a time interval of every two times of training is 15-20 min. During the test, an entry point is selected in a semi-random mode each time; and the mice are held up by hands of an experimenter to face the pool wall and then gently put into the water. During every training, if the mice can find the platform within 60 s, retention time exceeding 2 s on the platform serves as a platform finding success mark, and time used from entry to platform finding success of the mice is recorded as the latency; and if the mice cannot find the platform within 60 s, the mice are guided by the experimenter to the platform by hands, and the latency is recorded as 60 s. After climbing on the platform, the mice are enabled to stay on the platform for 10 s to conduct spatial learning and memorizing according to reference objects in 4 quadrants and to decrease tension of the mice. The mean of the latency per day is calculated to evaluate spatial memory acquirement ability of the animals.

Space exploration test: the platform is removed; each mouse is tested once; and test time is 60 s. The mouse is gently put into water facing the edge of the pool wall in the selected SE quadrant; and spatial memory ability of the animals is evaluated through the times of the animals crossing the original platform within 60 s, mouse swimming paths, and swimming distance ratios and time ratios of the original platform quadrants (i.e., ratios of swimming distances and time of the original platform quadrants of the animals accounting for the total swimming distances and total time).

### 5.4 Statistical Method

The data is analyzed by GraphPad Prism 8.02 software (Graph Pad Software, Inc., San Diego, California, USA). Before parameter test of the tests of all the groups, the data is subjected to normality test (Kolmogorov-Smirnov test) and homogeneity test for variance (Levene test). Except for the escape latency of the water maze training period, every two of all tested parameters should be compared among the groups by unpaired T test (one-tailed test); and a test level is set as p<0.05. The escape latency of the water maze training period should be tested by two-factor analysis of variance; post-test should use Bonferroni's multiple comparisons test; and a test level is set as p<0.05.

### 5.5 Test Results

### 5.5.1 Object Recognition Test

TABLE 5-1
Effect of the medicine in the present invention
on object recognition behaviors of mice (x ± SE)

| | | | Number of | |
|---|---|---|---|---|
| Group | Dose | | animals | Recognition index |
| Control group | — | | 15 | 0.5814 ± 0.06147 |
| Model group | — | | 14 | 0.422 ± 0.02834* |
| Positive medicine group | 26 | μg/kg | 15 | 0.5443 ± 0.03220# |
| Kaixin powder group | 2.5 | g/kg | 15 | 0.5012 ± 0.03547# |
| Qiongyugao group | 2.5 | g/kg | 15 | 0.4831 ± 0.03112# |
| Low-dose group | 1.25 | g/kg | 15 | 0.5081 ± 0.04122# |
| Medium-dose group | 2.5 | g/kg | 15 | 0.5378 ± 0.04617# |
| High-dose group | 5.0 | g/kg | 15 | 0.5411 ± 0.03555# |

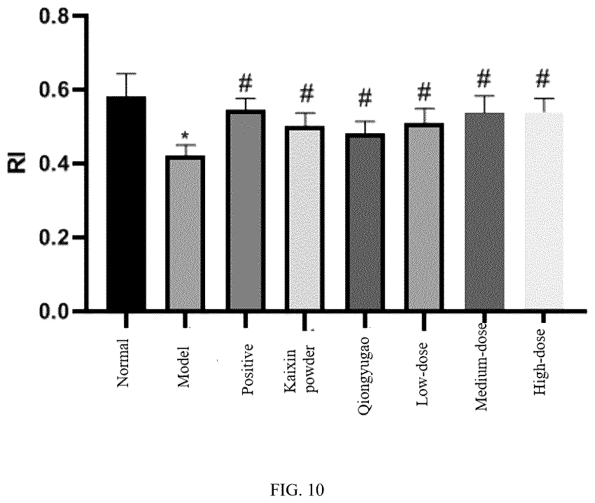

As shown in Table 5-1 and FIG. 10, the recognition index in the model group is significantly decreased compared with the normal control group, while the recognition index in the positive medicine group, the low-dose group, the medium-dose group and the high-dose group after intragastric administration is significantly increased. It is indicated that, the declarative memory ability of the mice in the model group is decreased; and the declarative memory ability of stressed mice in the positive medicine group, the Kaixin powder group, the Qiongyugao group, the low-dose group, the medium-dose group and the high-dose group after administration may be improved. Moreover, at the same dose, the recognition index of the medium-dose group of the medicine in the present invention is higher than that of the Kaixin powder group and the Qiongyugao group; and the difference is significant.

### 5.5.2 Step-Down Test

TABLE 5-2-1
Effect of the medicine in the present invention
on latency in step-down test of mouse (x ± SE)

| | | | Number of | |
|---|---|---|---|---|
| Group | Dose | | animals | Latency (s) |
| Control group | — | | 15 | 117.45 ± 30.35 |
| Model group | — | | 14 | 43.43 ± 10.32* |
| Positive medicine group | 26 | μg/kg | 15 | 114.96 ± 28.74# |
| Kaixin powder group | 2.5 | g/kg | 15 | 84.41 ± 24.43 |
| Qiongyugao group | 2.5 | g/kg | 15 | 72.63 ± 25.73 |
| Low-dose group | 1.25 | g/kg | 15 | 106.53 ± 22.911# |
| Medium-dose group | 2.5 | g/kg | 15 | 103.37 ± 27.12# |
| High-dose group | 5.0 | g/kg | 15 | 123.53 ± 10.50# |

TABLE 5-2-2
Effect of the medicine in the present invention on
error times in step-down test of mouse (x ± SE)

| | | | Number of | |
|---|---|---|---|---|
| Group | Dose | | animals | Error times |
| Control group | — | | 15 | 1.1783 ± 0.1975 |
| Model group | — | | 14 | 2.8467 ± 0.3057* |
| Positive medicine group | 26 | μg/kg | 15 | 1.7417 ± 0.2836# |
| Kaixin powder group | 2.5 | g/kg | 15 | 2.3132 ± 0.2143 |
| Qiongyugao group | 2.5 | g/kg | 15 | 2.5147 ± 0.2981 |
| Low-dose group | 1.25 | g/kg | 15 | 2.3350-0.4528 |
| Medium-dose group | 2.5 | g/kg | 15 | 1.8407 ± 0.3571# |
| High-dose group | 5.0 | g/kg | 15 | 1.7190-0.5535# |

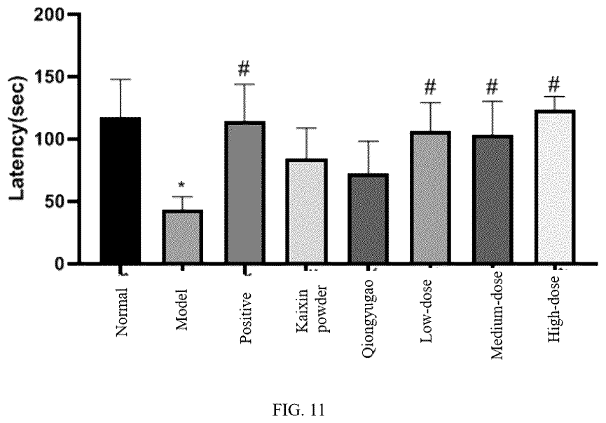

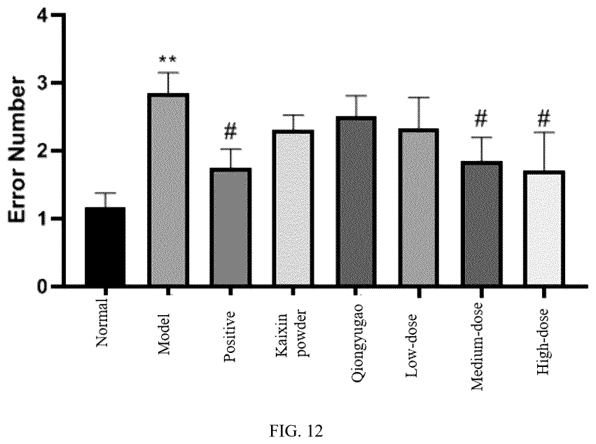

As shown in Table 5-2-1, Table 5-2-2 and FIGS. 11-12, the latency in the model group is significantly shortened compared with the control group, while the error times are significantly increased; and after intragastric administration, the latency in the positive medicine group, the low-dose group, the medium-dose group and the high-dose group is significantly prolonged compared with the model group, and is dose-dependent. Moreover, compared with the model group, the Kaixin powder group and the Qiongyugao group have no significance of difference. The error times in the positive medicine group, the medium-dose group and the high-dose group are significantly decreased. It is indicated that, the mice in the model group have step-down test memory ability impairment; and step-down test memory impairment may be improved in the positive medicine group, the medium-dose group and the high-dose group.

### 5.5.3 Water Maze Test

TABLE 5-3-1
Effect of the medicine in the present invention on escape latency
in training period of water maze test of mouse (x ± SE)

| | | | Number | | | | |
|---|---|---|---|---|---|---|---|
| | | | of | | | | |
| Group | Dose | | animals | Day 1 (s) | Day 2 (s) | Day 2 (s) | Day 4 (s) |
| Control group | — | | 15 | 54.5 ± 3.11 | 44.1 ± 2.42 | 36.3 ± 2.40 | 37.5 ± 2.41 |
| Model group | — | | 14 | 63.2 ± 2.28 | 54.2 ± 2.97 | 52.4 ± 2.33 | 57.7 ± 2.17* |
| Positive | 26 | μg/kg | 15 | 61.6 ± 2.76 | 53.3 ± 2.28 | 54.0 ± 2.49 | 52.9 ± 2.21 |
| medicine | | | | | | | |
| group | | | | | | | |
| Kaixin | 2.5 | g/kg | 15 | 56.5 ± 2.17 | 54.2 ± 2.56 | 51.3 ± 2.17 | 49.6 ± 2.30 |
| powder group | | | | | | | |
| Qiongyugao | 2.5 | g/kg | 15 | 54.8 ± 2.68 | 49.2 ± 2.71 | 47.5 ± 3.04 | 46.84 ± 2.49 |
| group | | | | | | | |
| Low-dose | 1.25 | g/kg | 15 | 60.3 ± 2.23 | 50.50 ± 2.92 | 48.96 ± 3.24 | 45.76 ± 2.56 |
| group | | | | | | | |
| Medium-dose | 2.5 | g/kg | 15 | 58.3 ± 2.31 | 56.5 ± 2.12 | 55.6 ± 2.69 | 49.5 ± 2.44 |
| group | | | | | | | |
| High-dose | 5.0 | g/kg | 15 | 52.9 ± 2.16 | 52.9 ± 2.38 | 53.2 ± 2.89 | 49.0 ± 2.24 |
| group | | | | | | | |

TABLE 5-3-2
Effect of the medicine in the present invention on escape latency
in exploration period of water maze test of mouse (x ± SE)

| | | | Number of | |
|---|---|---|---|---|
| Group | Dose | | animals | Escape latency (s) |
| Control group | — | | 15 | 22.06 ± 3.87 |
| Model group | — | | 14 | 43.41 ± 4.16* |
| Positive medicine | 26 | μg/kg | 15 | 25.10 ± 3.89# |
| group | | | | |
| Kaixin powder group | 2.5 | g/kg | 15 | 36.12 ± 3.24 |
| Qiongyugao group | 2.5 | g/kg | 15 | 39.57 ± 2.83 |
| Low-dose group | 1.25 | g/kg | 15 | 34.48 ± 3.58# |
| Medium-dose group | 2.5 | g/kg | 15 | 26.96 ± 4.15# |
| High-dose group | 5.0 | g/kg | 15 | 25.10 ± 4.23# |

TABLE 5-3-3
Effect of the medicine in the present invention
on target quadrant retention time in exploration
period of water maze test of mouse (x ± SE)

| | | | Number of | Target quadrant |
|---|---|---|---|---|
| Group | Dose | | animals | retention time (s) |
| Control group | — | | 15 | 18.56 ± 0.67 |
| Model group | — | | 14 | 14.25 ± 1.23* |
| Positive medicine | 26 | μg/kg | 15 | 15.34 ± 0.94 |
| group | | | | |
| Kaixin powder | 2.5 | g/kg | 15 | 15.45 ± 0.54 |
| group | | | | |
| Qiongyugao | 2.5 | g/kg | 15 | 14.89 ± 0.61 |
| group | | | | |
| Low-dose group | 1.25 | g/kg | 15 | 14.21 ± 0.52 |
| Medium-dose | 2.5 | g/kg | 15 | 15.67 ± 0.61 |
| group | | | | |
| High-dose group | 5.0 | g/kg | 15 | 16.88 ± 0.97# |

TABLE 5-3-4
Effect of the medicine in the present invention on target quadrant
path in exploration period of water maze test of mouse (x ± SE)

| | | | Number of | Target quadrant |
|---|---|---|---|---|
| Group | Dose | | animals | path (mm) |
| Control group | — | | 15 | 2934 ± 172.1 |
| Model group | — | | 14 | 2641 ± 208.0 |
| Positive medicine | 26 | μg/kg | 15 | 2956 ± 191.4 |
| group | | | | |
| Kaixin powder | 2.5 | g/kg | 15 | 2727 ± 202.1 |
| group | | | | |
| Qiongyugao group | 2.5 | g/kg | 15 | 2136 ± 198.4 |
| Low-dose group | 1.25 | g/kg | 15 | 2758 ± 156.7 |
| Medium-dose | 2.5 | g/kg | 15 | 2503 ± 173.2 |
| group | | | | |
| High-dose group | 5.0 | g/kg | 15 | 2904 ± 137.9 |

TABLE 5-3-5
Effect of the medicine in the present invention
on number of platform crossing times in exploration
period of water maze test of mouse (x ± SE)

| | | | Number of | Number of platform |
|---|---|---|---|---|
| Group | Dose | | animals | crossing times |
| Control group | — | | 15 | 3.467 ± 0.3922 |
| Model group | — | | 14 | 1.223 ± 0.4120* |
| Positive medicine | 26 | μg/kg | 15 | 2.667 ± 0.5120# |
| group | | | | |
| Kaixin powder group | 2.5 | g/kg | 15 | 2.164 ± 0.4518# |
| Qiongyugao group | 2.5 | g/kg | 15 | 1.941 ± 0.6127 |
| Low-dose group | 1.25 | g/kg | 15 | 1.806 ± 0.4420 |
| Medium-dose group | 2.5 | g/kg | 15 | 2.240 ± 0.6211# |
| High-dose group | 5.0 | g/kg | 15 | 2.847 ± 0.5224# |

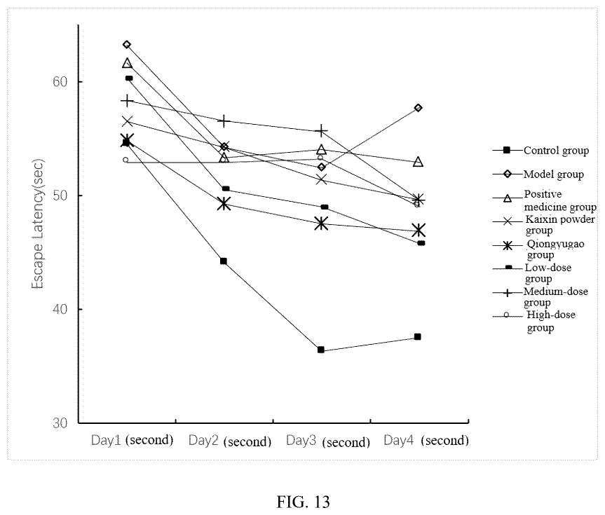

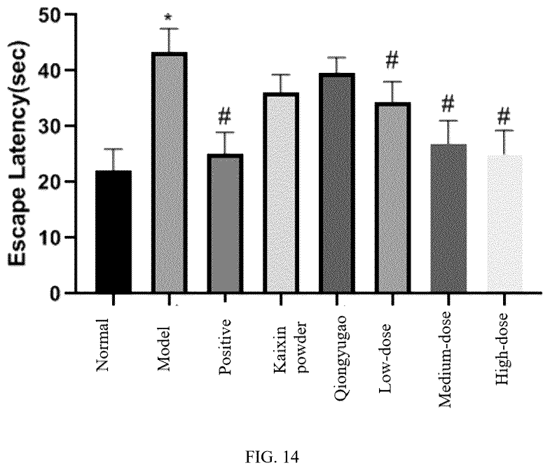

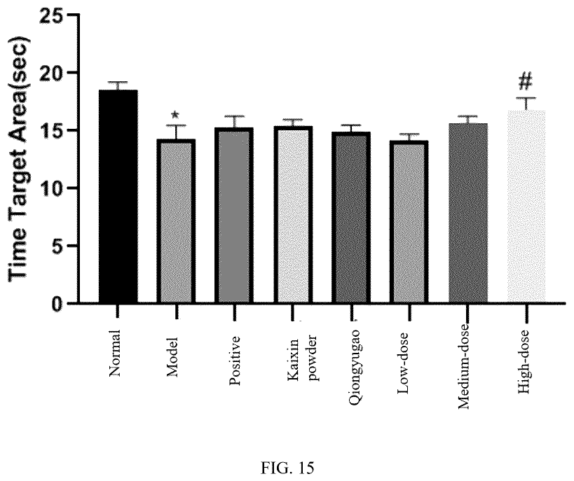

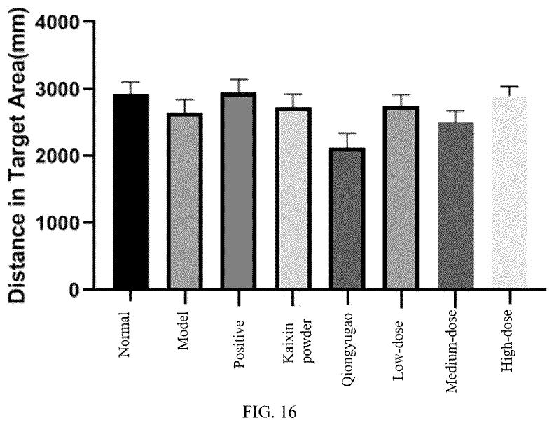

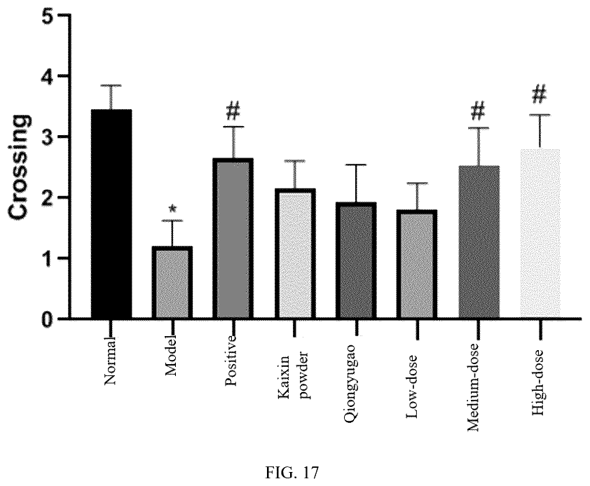

As shown in Tables 5-3-1, 5-3-2, 5-3-3, 5-3-4 and 5-3-5 and FIGS. 13-17, the escape latency on Day 4 of the training period of the water maze test in the model group is significantly prolonged compared with the control group, which indicates that the mice in the model group have memory storage ability impairment.

The escape latency in the exploration period of the water maze test in the model group is significantly prolonged compared with the control group, while the target quadrant retention time and the number of platform crossing times in the model group are significantly decreased compared with those in the control group, which indicates that the mice in the model group have memory retrieval ability impairment. After the intragastric administration, the escape latency in the positive medicine group, the medium-dose group and the high-dose group is significantly shortened compared with that in the model group; and the number of platform crossing times are significantly increased compared with the model group, which indicates that the spatial memory retrieval ability impairment of the mice may be improved after administration in the positive medicine group, the medium-dose group and the high-dose group.

### 5.6 Conclusion

Through the learning and memorizing triplet (Morris water maze+object recognition+step-down test) mouse behavior model, it is preliminarily understood that, memory impairment caused by scopolamine can be increased to some extent in the medium-dose group and the high-dose group of the medicine in the present invention. Moreover, at the same dose, the improvement effect is better than that of the Kaixin powder group and the Qiongyugao group.

### 6. Typical Cases

### Typical Case 1:

Li XX, female, 72, suffers from insomnia for many years, and often sleeps for 3-4 hours at night. In recent two years, Li XX has significantly decreased short-term memory, repeated discourse and restrained eyesight. While seeing a doctor on Mar. 18, 2021, Li XX was diagnosed with early Alzheimer's disease, having a clinical dementia rating CDR value of 0.8 and mild cognitive impairment MMSE22. 2 bags of the medicine in the present invention (granules in (7) of embodiment 2) were taken per day; each bag was 10 g; and the medicine was taken twice a day. After the medicine was taken for 2 months, relatives of the patient told that, the patient had richer facial expressions, better mental state and significantly improved sleep, can sleep for 5-7 hours, and has improved memory.

### Typical Case 2:

Shi XX, male, 76, has symptoms of impatience, irritability, dreaminess, early awakening and speech disorder and often get lost. While seeing a doctor on Apr. 20, 2021, Shi XX was diagnosed with moderate Alzheimer's disease, having a clinical dementia rating CDR value of 2.0 and mild cognitive impairment MMSE16. 2 bags of the medicine in the present invention (granules in (7) of embodiment 2) were taken per day; each bag was 10 g; and the medicine was taken twice a day. After the medicine was taken for 3 months, relatives of the patient told that, the patient had better mental state, decreased times of impatience and irritability and improved sleep, roughly remembered the way home, and had improved expressions.

### 7. Conclusion

In combination with in-vitro pharmacodynamic tests, in-vivo animal experiments and the typical cases, it may be predicted that the medicine in the present invention has an excellent effect of treating Alzheimer's disease.

The above description of the disclosed embodiments enables those skilled in the art to realize or use the present invention. Many modifications made to these embodiments will be apparent to those skilled in the art. General principles defined herein can be realized in other embodiments without departing from the spirit or scope of the present invention. Therefore, the present invention will not be limited to these embodiments shown herein, but will conform to the widest scope consistent with the principles and novel features disclosed herein.

What is claimed is:

1. A traditional Chinese medicine compound extract for treating Alzheimer's disease, comprising the following raw materials in parts by weight: 4-10 parts of ginseng, 6-12 parts of Cistanche deserticola, 4-10 parts of Salvia miltiorrhiza, 6-12 parts of Radix polygalae, 4-10 parts of Rhizoma acori tatarinowii, 6-12 parts of dried rehmannia root and 8-16 parts of Poria cocos.

2. The traditional Chinese medicine compound extract for treating Alzheimer's disease according to claim 1, comprising the following raw materials in parts by weight: 6 parts of ginseng, 10 parts of Cistanche deserticola, 6 parts of Salvia miltiorrhiza, 10 parts of Radix polygalae, 6 parts of Rhizoma acori tatarinowii, 10 parts of dried rehmannia root and 12 parts of Poria cocos.

3. A preparation method of the traditional Chinese medicine compound extract for treating Alzheimer's disease, specifically comprising the following steps:
(1) weighing
weighing various raw materials according to weight parts of the traditional Chinese medicine compound extract for treating Alzheimer's disease according to claim 1;
(2) alcohol extraction
mixing ginseng, Cistanche deserticola, Salvia miltiorrhiza and Radix polygalae; then adding ethanol having a mass concentration of 50-80%; performing heating reflux extraction for 1-3 times for 1-3 h each time; collecting alcohol extraction medicine liquid and alcohol extraction medicine residue respectively; and concentrating the alcohol extraction medicine liquid to obtain an alcohol extraction concentrated solution for later use;
(3) extraction and inclusion of volatile oil
soaking Rhizoma acori tatarinowii in water for 1-4 h; then extracting volatile oil for 1-10 h; collecting Rhizoma acori tatarinowii volatile oil, a Rhizoma acori tatarinowii extracting solution and Rhizoma acori tatarinowii medicine residue respectively for later use; adding the Rhizoma acori tatarinowii volatile oil into ethanol and dissolving to obtain an oil-ethanol mixture for later use; adding hydroxypropyl-beta-cyclodextrin into water and uniformly mixing; then slowly dropwise adding the oil-ethanol mixture into the solution; performing ball milling or colloid milling; and performing vacuum drying to obtain a Rhizoma acori tatarinowii inclusion compound for later use;
(4) water extraction
mixing the alcohol extraction medicine residue, the Rhizoma acori tatarinowii medicine residue, dried rehmannia root and Poria cocos; then adding water into the mixture; performing heating reflux extraction for 1-3 times for 1-3 h each time; collecting water extraction medicine liquid; concentrating the water extraction medicine liquid and the Rhizoma acori tatarinowii extracting solution; and placing the concentrated solution at room temperature to obtain water extraction concentrated solution;
(5) alcohol precipitation
adding ethanol into the water extraction concentrated solution for alcohol precipitation; refrigerating and centrifuging the solution; taking the supernatant; and concentrating the solution again to obtain a water extraction and alcohol precipitation concentrated solution for later use;
(6) concentrating and drying
mixing the alcohol extraction concentrated solution and the water extraction and alcohol precipitation concentrated solution; continuously concentrating the mixed solution; and performing vacuum drying or spray drying to obtain a mixed extract for later use;
(7) finished product preparation
uniformly mixing the Rhizoma acori tatarinowii inclusion compound and the mixed extract; grinding and sieving the mixture; adding an excipient into the mixture to make a preparation, thereby obtaining the medicine for treating the Alzheimer's disease;
wherein types of the preparation comprise pills, tablets, granules or decoction.

4. The preparation method of the traditional Chinese medicine compound extract for treating Alzheimer's disease according to claim 3, wherein in the step (2), the mass concentration of the ethanol is 60%; the frequency of heating reflux extraction is twice; an addition amount of the ethanol each time is 8 times that of the weight; and heating reflux extraction time for each time is 2 h.

5. The preparation method of the traditional Chinese medicine compound extract for treating Alzheimer's disease according to claim 4, wherein in the step (2), a concentration temperature is 55-75° C.; and specific gravity of the concentrated solution is 1.05-1.10/50° C.

6. The preparation method of the traditional Chinese medicine compound extract for treating Alzheimer's disease according to claim 3, wherein in the step (3), an addition amount of the water is 8 times that of the weight; soaking time is 2 h; and volatile oil extraction time is 6 h.

7. The preparation method of the traditional Chinese medicine compound extract for treating Alzheimer's disease according to claim 3, wherein in the step (3), a volume ratio of the Rhizoma acori tatarinowii extracting solution to the ethanol is 1:1; a mass ratio of the hydroxypropyl-beta-cyclodextrin to the water to the oil-ethanol mixture is 15:7.5:1; ball milling or colloid milling time is 1 h; and a vacuum drying temperature is 40° C.

8. The preparation method of the traditional Chinese medicine compound extract for treating Alzheimer's disease according to claim 3, wherein in the step (4), the frequency of heating reflux extraction is twice; an addition amount of the water each time is 6 times that of the weight; heating reflux extraction time for each time is 1 h; a concentration temperature is 60-90° C.; and specific gravity of the concentrated solution is 1.05-1.10/60° C.

9. The preparation method of the traditional Chinese medicine compound extract for treating Alzheimer's disease according to claim 3, wherein in the step (5), the ethanol is added until alcohol content is 60%; the solution is continuously concentrated until specific gravity of the concentrated solution is 1.05-1.10/60° C.; and refrigeration time is 24 h.

10. The preparation method of the traditional Chinese medicine compound extract for treating Alzheimer's disease according to claim 3, wherein in the step (6), the solution is continuously concentrated until specific gravity of the concentrated solution is 1.10-1.15/60° C.; a vacuum drying temperature is 40-50° C.; and the temperature of a heating tube for spray drying is 120-130° C.; in the step (7), the type of the preparation is a granule.
